(19) United States
(12) Patent Application Publication
(10) Pub. No.: US 2004/0106566 A1
(43) Pub. Date: Jun. 3, 2004

(54) RNA-splicing and processing-directed gene silencing and the relative applications thereof

(76) Inventors: Shi-Lung Lin, Alhambra, CA (US); Shao-Yao Ying, San Marino, CA (US)

Correspondence Address: Shi-Lung Lin, 731 South Chapel Avenue, Apt# F, Alhambra, CA, 91801, US

(21) Appl. No.: 10/439,262

(22) Filed: May 15, 2003

Related U.S. Application Data
(60) Provisional application No. 60/381,651, filed on May 17, 2002.
(60) Provisional application No. 60/411,062, filed on Sep. 16, 2002.
(60) Provisional application No. 60/418,405, filed on Oct. 12, 2002.

(51) Int. Cl. A61K 48/00; C12N 15/85

(52) U.S. Cl. 514/44; 435/455

(57) ABSTRACT

The present invention relates to a method for generating a recombinant gene composition, which is able to elicit specific gene silencing effects through RNA splicing and/or processing mechanisms, and the relative utilization thereof. The recombinant gene molecule so generated is useful not only for delivering desirable gene function into the transfected cells thereof but also for suppressing undesirable gene function in the transfected cells, respectively or simultaneously. Furthermore, the derivative products of this novel recombinant gene have multiple utilities in probing gene function, validating drug target, and treating as well as preventing gene-related diseases.

### CLAIM OF THE PRIORITY

The present application claims priority to U.S. Provisional Application Serial No. 60/381,651 filed on May 17, 2002, entitled “IN VIVO PRODUCTION OF SPECIFIC RNA MOLECULES BY RNA SPLICING”, U.S. Provisional Application Serial No. 60/411,062 filed on Sep. 16, 2002, entitled “VECTOR-BASED GENE MODULATION USING RNA SPLICING MECHANISM” and U.S. Provisional Application Serial No. 60/418,405 filed on Oct. 12, 2002, entitled “COMBINATIONAL THERAPY FOR HIV ERADICATION AND VACCINATION”, which are hereby incorporated by reference as if fully set forth herein.

### FIELD OF THE INVENTION

The present invention relates to a method and composition for generating an artificially recombinant gene molecule capable of inducing specific gene silencing effects through cellular RNA splicing and/or processing mechanisms, and the relative utilization thereof. The recombinant gene so generated is useful not only for delivering desired gene function into the transfected cells thereof but also for suppressing unwanted gene function in the transfected cells. Furthermore, the derivative products of this novel recombinant gene have utilities in probing gene function, validating drug target, and treating as well as preventing gene-related diseases.

### BACKGROUND OF THE INVENTION

Therapeutic intervention of a human disease can be achieved by targeting specific disease-associated or causing genes such as replacing impaired or missing gene by introducing functional gene in a gene therapy, suppressing gene function by antisense oligonucleotide against specific disease gene, antibody therapeutics against disease target or small molecule drug as antagonist or agonist agent for a drug target. Recent advent in RNA interference (RNAi) technologies provides novel agents in double-stranded short-interfering RNA (siRNA) (Elbashir et.al. (2001) Nature 411: 494-498) and doxyribonucleotidylated-RNA interfering (D-RNAi) (Lin et.al. (2001) Biochem. Biophys. Res. Commun. 281: 639-644) molecules that may have great therapeutical utilities in human. The RNAi elicits post-transcriptional gene silencing (PTGS) phenomena capable of knocking down specific gene expression with high potency at a few nanomolar dosage, which has been proven to be much less toxic than traditional antisense gene therapies. Based on prior studies, the siRNA-induced gene silencing effect usually lasts one week, while that of D-RNAi can sustain up to one month. These phenomena appear to evoke an intracellular sequence-specific RNA degradation process, affecting all highly homologous transcripts, called cosuppression. It has been proposed that such cosuppression results from the generation of small interfering RNA products (21˜25 nucleotide bases) by the activities of an RNA-directed RNA polymerase (RdRp) and/or a ribonuclease (RNase) on aberrant RNA templates, which are derived from the transfection of nucleic acids or viral infection (Grant, S. R. (1999) Cell 96, 303-306; Lin et.al (2001) Current Cancer Drug Targets 1: 241-247).

Although PTGS/RNAi phenomena appear to offer a potential avenue for inhibiting gene expression, their applications have not been demonstrated to work constantly in higher vertebrates and, therefore, the widespread use thereof in higher vertebrates is still questionable. For example, the findings of RNAi effects are based on the transfection use of double-stranded RNA (dsRNA), which have shown to cause interferon-induced non-specific RNA degradation in mammalian cells (Stark et.al. (1998) Annu. Rev. Biochem. 67: 227-264; Elbashir supra; U.S. Pat. No. 4,289,850 to Robinson; and U.S. Pat. No. 6,159,714 to Lau). Such an interferon-induced cellular response usually reduces the specificity of RNAi-associated gene silencing effects and may cause a severe cytotoxic side-effect to the transfected cells (Stark et.al. supra; Elbashir supra). Especially in mammalian cells, it has been noted that the gene silencing effects of dsRNA-mediated RNAi phenomena are repressed by the interferon-induced global RNA degradation when the dsRNA size is larger than 25 base-pairs (bp). Although the transfection of short interfering RNA (siRNA) or microRNA (mRNA) sized less than 21 bp can overcome the interferon-associated problems, unfortunately for therapeutic use, this limitation in size impairs the usefulness of siRNA because it would be difficult to deliver such small and unstable dsRNA construct in vivo due to the high dsRNase activities of our bodies (Brantl S. (2002) Biochimica et Biophysica Acta 1575, 15-25).

Other types of therapeutics, such as antisense oligonucleotide-based or ribozyme molecule-based molecules, target the undesirable messenger RNA (mRNA) transcript of gene in hoping of suppressing the undesired gene function. These therapeutic interventions inhibiting the expression of a gene or gene function by ways of blocking gene product translation, causing fast gene transcript (mRNA) degradation or preventing pre-mRNA maturation such as breakdown of pre-mRNA, hnRNA, tRNA, rRNA and other RNP molecules. This type of therapies holds great promise in disease therapy and diagnosis. In fact, the antisense technology has been successfully applied to cancer and genetic research in vitro as well as in vivo (Jen et.al. (2000) Stem Cells 18: 307-319; Ying et.al. (1999) Biochem. Biophys. Res. Commun. 265: 669-673). The antisense technology involves the intracellular transduction of an oligonucleotide sequence that is capable of complementarily binding to a targeted mRNA in cells and thus inhibits the expression of the mRNA. However, many problems remain due to the low efficacy and high cytotoxicity of all antisense technologies. For example, single-stranded DNA antisense oligonucleotides exhibit only short-term effectiveness and are usually toxic at the doses required for biological effectiveness. Similarly, the use of single-stranded antisense RNAs has also proven to be ineffective due to its fast degradation and structural instability.

As a common knowledge in the gene therapy field, a functional gene is preferably delivered into a cell or human being by gene-expressing vector vehicles, including retroviral vector, lentiviral vector, adenoviral vector, adeno-associated viral (AAV) vector and so on. The desirable gene function so introduced into the cells is activated through gene transcription and subsequently translation to form a functional polypeptide or protein for compensating the missing gene function or competing with the normal function of relative gene homologues. The main purpose of these vector-based approaches is to maintain long-term gene modulation. However, previous vector-based technologies, such as antisense-expressing and dominant-negative gene silencing vectors, have been shown to cause tedious works in target selection and usually provide inconsistent efficacy (Jen, supra). On the other hand, the utilization of siRNA-expressing vectors has been reported to offer stable efficacy and lower interferon-induced toxicity for RNAi induction (Tuschl et.al. (2002) Nat Biotechnol. 20: 446-448). Although prior arts (Miyagishi et.al. (2002) Nat Biotechnol 20: 497-500; Lee et.al. (2002) Nat Biotechnol 20: 500-505; Paul et.al. (2002) Nat Biotechnol 20: 505-508) attempting to use this approach have succeeded in maintaining constant RNAi efficacy, their delivery strategy did not provide global effectiveness for the targeted cell population. Moreover, the requirement of using type III RNA polymerase (Pol-III) promoters, such as U6 and H1, for siRNA generation is another drawback. Because the read-through and unreliable side-effects of a Pol-III transcription machinery occurs on a short transcription template without proper termination codon, cellular type-III RNA polymerases occasionally synthesize RNA products longer than desired siRNA and then cause unexpected interferon cytotoxicity (Geiduschek et.al. (2001) J. Mol Biol 310: 1-26; Schramm et.al. (2002) Genes Dev 16: 2593-2620). Furthermore, despite the widespread existing of Pol-III promoters in a variety of human cells, the activity of type-III RNA transcription machinery may not be very active in some cell types of interest. These disadvantages hinder the use of vector-directed gene silencing for therapeutical purposes.

In sum, in order to increase the delivery stability, spreading coverage and multiplication of high efficient gene silencing effects, a better induction and maintenance strategy is highly desired. Therefore, there remains a need for an effective, stable and reliable gene modulation method as well as agent composition for inhibiting and/or expressing gene function through PTGS/RNAi mechanisms.

### SUMMARY OF THE INVENTION

Research based on transcriptome, an assembly of gene exons, is fully described throughout the literature, taking the fate of a spliced intron to be digested for granted (Clement et.al. (1999) RNA 5: 206-220; Sittler et.al. (1987) J. Mol Biol 197: 737-741). Is it true that the non-protein-coding nucleotide sequence of a gene such as intron is destined to be a metabolic waste without function or there is a function for it which has not yet been discovered? Our present invention provides a novel composition and method for disclosing the profound function of intron in the aspect of gene regulation and its relative utilities thereof. Based on RNA splicing and processing mechanisms, we have designed a recombinant gene construct containing splicing-competent intron(s), which is able to inhibit the function of a gene that is homologous to the intron when it is released from the recombinant gene transcript by intracellular splicing and/or processing machinery. The spliced exons of the recombinant gene will be linked together and become a mature RNA molecule that is useful in generating desired gene function of an impaired, missing or marker gene in eukaryotic cells. Without being bound by any particular theory, the method for generating and using the present invention relies on the genetic engineering of RNA splicing/processing apparatus to form an artificial intron with at least a desired RNA insert and the incorporation of the intron into a recombinant gene for the transcription of the intron-containing recombinant gene transcripts (pre-mRNA) in a cell During mRNA maturation, the desired RNA insert molecule will be released by splicing/processing machinery and then induces desired gene silencing effects, while the rest exon parts of the spliced recombinant gene transcript can be linked together to form mature mRNA for the expression of desirable gene function.

In accordance with the present invention, the mature RNA molecule formed by the linkage of exons may be useful in conventional gene therapy to replace impaired or missing gene function, or to increase specific gene expression. Additionally, the present invention provide novel compositions and means in producing intracellular gene silencing molecules by way of RNA splicing and/or processing mechanisms to elicit either antisense oligonucleotide effect or RNA interfering (RNAi) effect useful for inhibiting gene function. The splicing-and/or processing-mediated gene silencing molecules, such as antisense RNA and RNAi constructs, resulting from the present invention is preferably used to target a gene selected from the group consisting of pathogenic nucleic acids, viral genes, mutated genes, oncogenes and any other types of functional as well as non-functional genes.

In one preferred embodiment (FIG. 1), the present invention provides a method for suppressing gene function or gene silencing, comprising the steps of: a) providing: i) a substrate expressing a targeted gene, and ii) an expression-competent composition comprising a recombinant gene capable of producing specific RNA transcript, which is in turn able to generate pre-designed gene silencing molecules through RNA splicing and/or processing mechanisms to knock down or silence the expression of the targeted gene in the substrate; b) treating the substrate with the composition under conditions such that the targeted gene expression in the substrate is inhibited. The substrate can express the targeted gene either in cell, ex vivo or in vivo. In one aspect, the RNA-splicing/processing-generated gene silencing molecule is an RNA insert located within the intron area of the recombinant gene and is capable of silencing a gene selected from the group consisting of pathogenic nucleic acids, viral genes, mutated genes, oncogenes and any other types of physiologically functional genes. Such RNA insert can also be artificially incorporated into the intron area of any kind of genes that are expressed in a cell. In principle, the molecular biological procedure for this kind of intron replacement in a gene is based on the same methodology used for the construction of an artificial recombinant gene demonstrated by the present invention (especially in Examples 2 and 3 and FIG. 1).

In another aspect, the artificial construct of a recombinant gene of the present invention is a mimicry to a pre-mature RNA (pre-mRNA) molecule. The recombinant gene template is consisted of two different parts: exon and intron. The exon part is spliced and ligated to form a functional gene for tracking the splicing activity. The intron part is spliced and further processed into a desired RNA molecule, serving as the aforementioned gene silencing molecule. The desired RNA molecule may be immediately flanked with at least one stem-loop structure comprising a sequence homologous to (A/U)UCCAAGGGGG motif for accurate splicing of the desired RNA molecule out of intron without further unwanted U4/U6 degradation. The 5′-end of an intron contains a splicing donor site homologous to either GTAAGAGK or GU(A/G)AGU motif, while the 3′-end is a splicing acceptor site that is homologous to either TACTWAY(N)mGWKSCYRCAG or CT(A/G)A(C/T)NG motif, and preferably m≧1. The adenosine “A” nucleotide of the CT(A/G)A(C/T)NG motif transcripts is part of (2′-5′)-linked branchpoint acceptor formed by (2′-5′)oligoadenylate synthetase in eukaryotic cells and the symbolic “N” nucleotide is either a nucleotide (ex. deoxyadenosine, deoxyguanosine, deoxycytidine, deoxythymine, deoxyuridine, riboxyadenosine, riboxyguanosine, riboxycytidine, riboxythymine and riboxyuridine) or an oligonucleotide, most preferably a T- and/or C-rich oligonucleotide. There could be a linker nucleotide sequence for the connection of the stem-loop to either the splicing donor or acceptor, or both.

In another preferred embodiment of the present invention (FIGS. 2-4), the recombinant gene composition can be cloned into an expression-competent vector. The expression-competent vector is selected from a group consisting of plasmid, cosmid, phagemid, yeast artificial chromosome, retroviral vectors, lentiviral vector, lambda vector, adenoviral (AMV) vector, adeno-associated viral (AAV) vector, hepatitis virus (HBV)-modified vector and cytomegalovirus (CMV)-related viral vectors. The strength of this strategy is in its deliverability through the use of viral infectious vectors, providing a stable and relatively long-term effect of specific gene silencing. Applications of the present invention include, without limitation, therapy by suppression of cancer-related genes, vaccination against potential viral genes, treatment of microbe-related genes, research of candidate molecular pathways with systematic knockout/knockdown of involved molecules, and high throughput screening of gene functions based on microarray analysis, etc. The present invention can also be used as a tool for studying gene function in physiological and therapeutical conditions, providing a composition and method for altering the characteristic of an eukaryotic cell. The cell can be selected from the group of cancerous, virus-infected, microbe-infected, physiologically diseased, genetically mutated, pathogenic cells and so on.

In one aspect, the recombinant gene, for example encoding an antisense RNA molecule as shown in FIG. 2, is generated by intracellular RNA splicing and/or processing mechanisms, ranged from a few oligonucleotide to a few hundred ribonucleotide bases in length. Such antisense RNA molecule effects antisense gene knockdown activity for suppressing targeted gene function in the cell. Alternatively, the antisense RNA molecule can bind to the sense strand of targeted gene transcripts to form long double-stranded RNA (dsRNA) for inducing interferon-associated cytotoxicity in order to kill the transfected cell, while the transfected cell is a substrate organism selected from the group of cancerous, virus-infected, microbe-infected, physiologically diseased, genetically mutated, pathogenic cells and so on. In another aspect, the present invention can be used in relation to posttranscriptional gene silencing (PTGS) technologies as a powerful new strategy in the field of gene therapy (FIGS. 3&4). The RNA splicing/processing-mediated cellular event produces small interfering RNA (siRNA), double-stranded RNA (dsRNA), microRNA (mRNA) or small hairpin RNA (shRNA) molecule, or their combination that is able to cause RNAi-like gene silencing phenomena. The siRNA/mRNA/shRNA so obtained is of 16 to 38 base pairs (bp), preferably of 19 to 25 bp. The siRNA/mRNA/shRNA molecule is desired to be constantly produced in the transfected cell by promoter-driven mRNA transcription machinery.

However, the expression of small-sized RNA molecule is usually impossible to be maintained in a cell by most of type II RNA polymerase (Pol-II)-mediated and viral promoters. Unlike a type-III RNA polymerase (Pol-III)-mediated U6 or H1 promoter, typical mRNA transcription generates a fairly large RNA transcript (>300 bases) which contains multiple copies of exon and intron sequences. The exon is the component parts of a functional gene transcript (mRNA), while the intron is thought to be unessential to the gene function of the exons. In principle, mRNA maturation requires the splicing of intron out of exon sequences and then the ligation of the exon sequences into one relatively mature mRNA. Therefore, based on this mRNA maturation procedure, a desired RNA molecule can be inserted into intron area for later releasing intracellularly by the splicing and/or processing mechanisms (FIG. 1). On the other hand, the exon sequences can be replaced by a reporter gene or gene marker, such as green fluorescin protein (GFP), luciferase, lac-Z, and their derivative homologues. The mRNA maturation of these tracking genes is useful for locating the desired RNA molecule, facilitating splicing accuracy and/or preventing unwanted degradation.

To produce small RNA sequences, such as siRNA, mRNA and shRNA, spliced from a pre-mRNA transcript of the present recombinant gene in a cell, an expression-competent vector may be needed for stable transfection and expression of the pre-mRNA molecule. The desired RNA molecule is released by the cell through promoter-driven mRNA transcription and then splicing/processing machinery. The expression-competent vector can be selected from a group consisting of plasmid, cosmid, phagemid, yeast artificial chromosome, retroviral vectors, lentiviral vector, lambda vector, AMV, CMV, AAV and Hepatitis-virus vectors. The expression of the pre-mRNA is driven by either viral or cellular RNA polymerase promoter(s) or both. For example, a lentiviral LTR promoter is sufficient to provide up to 5×10⁵ copies of pre-mature mRNA per cell. It is feasible to insert a drug-sensitive repressor in front of the lentiviral promoter in order to control the expression rate. The repressor can be inhibited by a chemical drug or antibiotics selected from the group of tetracycline, neomycin, ampicillin, etc.

The desired RNA molecule can be homologous to an RNA transcript or a part of the RNA transcript of a gene selected from the group consisted of fluorescent protein genes, luciferase genes, lac-Z genes, plant genes, viral genomes, bacterial genes, animal genes and human oncogenes. The homologous region of the desired RNA molecule is sized from about 17 to about 10,000 nucleotide bases, most preferably in between 19 to 2,000 bases. Alternatively, the desired RNA molecule is complementary to an RNA transcript or a part of the RNA transcript of a gene selected from the group consisted of fluorescent protein genes, luciferase genes, lac-Z genes, plant genes, viral genomes, bacterial genes, animal genes and human oncogenes. The complementary region of the desired RNA molecule is sized from about 17 to about 10,000 base pairs, most preferably in between 19 to 500 base pairs. The desired RNA molecule also could be the combination of the above molecule, such as a palindromic nucleotide sequence able to form hairpin conformation. The homology and/or complementarity rate is ranged from about 30˜100%, more preferably 35˜49% for a desired hairpin-RNA molecule and 90˜100% for both desired sense- and antisense-RNA molecules.

The present invention provides novel means of producing aberrant RNA molecules in cell as well as in vivo, especially such as siRNA/mRNA/shRNA compositions in vivo to induce PTGS/RNAi-associated phenomena. Hence, the present invention provides novel intracellular RNA generation and processing method for producing sense or antisense, long or short RNA molecules of pre-determined length and specificity. The desired RNA product after the intracellular splicing/processing procedure (SpRNAi) can be produced in single unit or in multiple units on a recombinant gene transcript of the present invention. Same or different spliced RNA molecules can be produced in either sense or antisense orientation in comparison to the mRNA transcript of an interesting gene. In certain case, spliced RNA molecules complementary to a gene transcript (mRNA) can be hybridized through intracellular formation of double-stranded RNA (dsRNA) for effecting either RNAi-related phenomena with short dsRNA or interferon-induced cytotoxicity with long >25 bp dsRNA. In other case, either small-interfering RNA (siRNA), microRNA (mRNA) or short-hairpin RNA (shRNA) molecules, or the combination thereof, can be produced as small spliced RNA molecules for induction of the PTGS/RNAi-associated gene silencing effects. The spliced siRNA/mRNA/shRNA molecule so obtained can be constantly produced by an expression-competent vector in vivo, thus, alleviate concerns of fast small dsRNA degradation. The spliced RNA obtained from cell culture can also be purified in vitro for generating either dsRNA or deoxyribonucleotylated RNA (D-RNAi) that is capable of inducing RNAi or PTGS phenomena respectively when the dsRNA is to be introduced into cells under non-vector basis.

### BRIEF DESCRIPTION OF THE DRAWINGS

The FIG. 1 depicts a novel strategy for producing desired RNA construct molecules in cells after RNA splicing event occurs. The oligonucleotide template of the desired RNA molecule is flanked with a RNA splicing donor and an acceptor site as the same as occurs in a natural intron. The template is inserted into a gene, which is expressed by type-II RNA polymerase (Pol-II) transcription machinery under the control of either Pol-II or viral RNA promoter. Upon intracellular transcription, the gene transcript so produced is subjected to RNA splicing and/or processing events and therefore releases the pre-designed, desired RNA molecule in the transfected cell. In certain case, the desired RNA molecule is an antisense RNA construct that can be served as antisense oligonucleotide probes for antisense gene therapy (FIG. 2). In other case, the desired RNA molecule can be of either sense or antisense orientation and possessing all element/domain sequences needed for polypeptide translation and termination (FIG. 3). The polypeptide or protein encoded by the desired RNA molecule will be useful in gene replacement therapy. In some other cases, the desired RNA molecule consists of small antisense and sense RNA fragments to function as double-stranded siRNA for RNAi induction (FIG. 3). In yet other cases, the desired RNA molecule is a small hairpin-like RNA construct capable of causing RNAi-associated gene silencing phenomena (FIG. 4). All the above desired RNA construct molecules are produced by the intracellular splicing events and named “SpRNAi” for convenience.

Referring particularly to the drawings for the purpose of illustration only and not limitation, there is illustrated:

FIG. 1 depicts the principal embodiment of SpRNAi-containing recombinant gene construct, construction and the relative applications thereof.

FIG. 2 depicts the first preferred embodiment of antisense RNA generation by spliceosome cleavage from retroviral (e.g. LTR) promoter-mediated precursor transcripts.

FIG. 3 depicts the second preferred embodiment of sense and antisense siRNA generation by spliceosome cleavage from viral (e.g. CMV or AMV) promoter-mediated precursor transcripts.

FIG. 4 depicts the third preferred embodiments of hairpin RNA generation by spliceosome cleavage from Pol-II (e.g. TRE or Tet response element) promoter-mediated precursor transcripts.

FIG. 5 depicts the microscopic results of Example 5, showing interference of green fluorescent protein (eGFP) expression in rat neuronal stem cells by various SpRNAi constructs made from Examples 2 and 3.

FIG. 6 depicts the western blotting results of Examples 5 and 6, showing interference of green fluorescent protein (eGFP) expression in rat neuronal stem cells by various SpRNAi constructs made from Examples 2 and 3.

FIG. 7 depicts the western blotting results of Examples 5 and 6, showing interference of integrin β1 (ITGb1) expression in human prostatic cancer LNCaP cells by various SpRNAi constructs made from Example 3.

FIG. 8 depicts the northern analysis of SpRNAi-induced cellular gene silencing against HIV-1 infection (n=3).

FIG. 9 depicts the potential differences between traditional PTGS/RNAi and current SpRNAi phenomena.

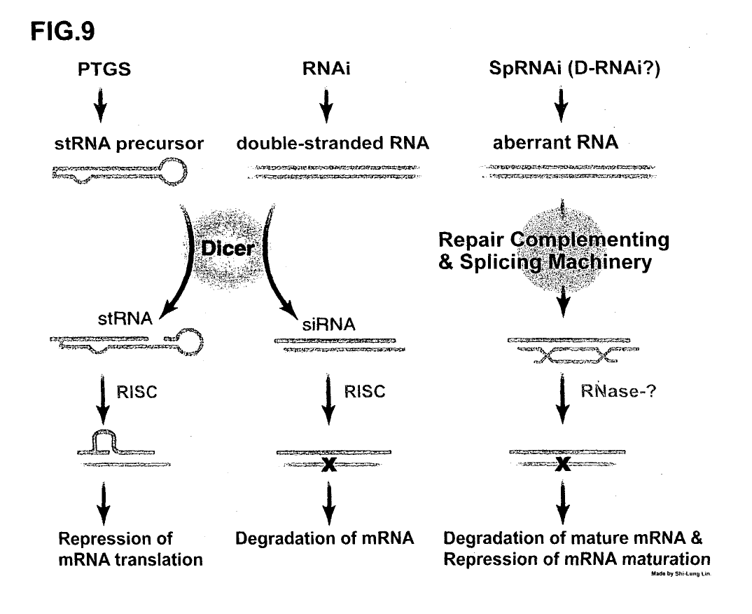

### DETAILED DESCRIPTION OF THE INVENTION

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

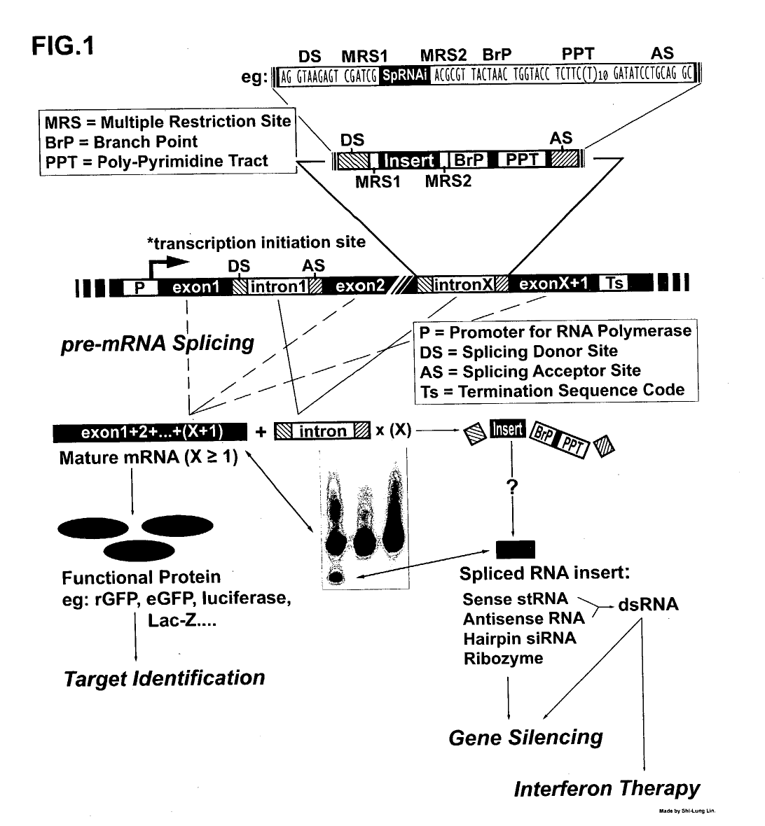

The present invention provides a novel composition and method for altering genetic characteristics of a cell. Without being bound by any particular theory, such alteration of cellular gene characteristics may be directed to a newly discovered PTGS-associated gene silencing phenomenon, triggered by the introduction of an artificially recombinant gene containing RNA splicing/processing-competent intron (SpRNAi) molecule into the cell. Generally, as seen in FIGS. 1 & 2-4, when the recombinant gene is transduced, transfected, or otherwise introduced by infection into the cell, small fragments of SpRNAi inserts may be produced by cleavage and processing of the RNA transcripts of the recombinant gene through intracellular interactions with spliceosome machinery. The freely released SpRNAi inserts can therefore induce posttranscriptional gene silencing (PTGS)— and/or RNA interference (RNAi)-like effects against targeted gene expression, and consequently the targeted gene transcript (mRNA) becomes degraded by RDE and/or RNase III endonucleases present in the cell Due to lack of mRNA of the targeted gene, no protein synthesis occurs, resulting in the silencing of the gene from which the mRNA was transcribed.

Similar to natural pre-messenger RNA (pre-mRNA) splicing/processing processes, the spliceosome machinery that catalyzes intron removal in the RNA transcript of our designed SpRNAi-inserted recombinant gene is formed by sequential assembly on selected SpRNAi regions of modular elements (snRNPs U1, U2 and U4/U6.U5 tri-snRNP) and numerous non-snRNP proteins. The methods for incorporation of these element-recognition sites into a SpRNAi intron are described in Examples 2 and 3. In brief, a sequential order of addition of the snRNPs has been proposed: first, recognition of the 5′-splicing junction (splicing donor site) by the U1 snRNP, then interaction of the branch-site sequence with the U2 snRNP, and finally, association of the U4/U6.U5 tri-snRNP to form an early splicing complex for precisely cleavage of the 5′-splicing junction. The 3′-splicing junction (splicing acceptor site) is cleaved by a late splicing complex formed by U5 and some unknown “late” splicing proteins after the release of the 5′-splicing junction. However, little is known on the protein/protein and RNA/protein interactions that bridge the U4/U6 and U5 snRNP components within an eukaryotic tri-snRNP, and knowledge on the binding sites of proteins on U4/U6 and U5 snRNPs remains limited as well.

Design of Artificially Recombined Genes for Testing Splicing-Directed Gene Silencing Effects.

Strategy for molecular analysis of RNA splicing/processing-directed gene silencing mechanisms was tested using an artificially recombined gene, termed SpRNAi-rGFP (FIG. 1). Genetic recombination of a splicing-competent intron (SpRNAi) into an intron-free red fluorescin gene (rGFP) was performed, providing splicing-directed gene silencing effects through pre-mRNA splicing and some unknown processing mechanisms. Although we showed here a model of gene silencing through pre-mRNA splicing, the same principle can be used for the design of gene silencing inserts working through pre-ribosomal RNA (pre-rRNA)-processing, which is mainly functioned by type-I RNA polymerase (Pol-I) transcription machinery. The splicing-competent intron is flanked with a donor (DS) and an acceptor (AS) splicing site, and contains at least one gene homologue insert, branch point (BrP) and poly-pyrimidine tract (PPT) in between the DS-AS sites for interacting with spliceosome machinery. Using low stringent northern blotting (middle bottom of FIG. 1), we were able to observe the release of 15˜45 bp intron-insert fragments from the designed SpRNAi-rGFP gene transcript (left), rather than an intron-free rGFP (middle) or a defective SpRNAi-rGFP (right) RNA without a functional splicing donor site, while spliced exons were linked to form mature RNA for rGFP protein synthesis. The “?” mark indicates an unknown mechanism for processing of a ˜120-bp intron to the small interfering intron-insert fragments. We have successfully tested short sense, short antisense and hairpin constructs of some gene homologue inserts for induction of specific gene silencing in various cell types.

As shown in FIG. 1, splicing-competent introns (SpRNAi) were synthesized and inserted into an intron-free red fluorescin gene (rGFP) that was mutated from the HcRed1 chromoproteins of Heteractis crispa. Since the inserted intron(s) disrupted the functional fluorescin structure of rGFP proteins, we were able to check the occurrence of intron splicing and rGFP-mRNA maturation through the reappearance of red fluorescent light emission at the 570-nm wavelength in a successfully transfected cell. Construction of SpRNAi was based on the natural structures of a pre-messenger RNA intron, consisting of spliceosome-dependent nucleotide components, such as donor and acceptor splicing sites in both ends for precise cleavage, branch point domain for splicing recognition, poly-pyrimidine tract for spliceosome interaction, linkers for connection of each major components and some artificially added multiple restriction/cloning sites for insert cloning, Based on prior studies, the donor splicing site is an oligonucleotide sequence either containing or homologous to the (5′-exon-AG)-(splicing point)-GTA(A/-)GAG(G/T)-3′ sequences (SEQ.ID.NO.1), including but not limited, 5′-AG GTAAGAGGAT-3′,5′-AG GTAAGAGT-3′,5′-AG GTAGAGT-3′,5′-AG GTAAGT-3′ and so on. The acceptor splicing site is an oligonucleotide sequence either containing or homologous to 5′-G(W/-)(T/G)(C/G)C(T/C)(G/A)CAG-(splicing point)-(G/C-3′-exon) sequences (while W is a pyrimidine A, T or U) (SEQ.ID.NO.2), including but not limited, 5′-GATATCCTGCAG G-3′,5′-GGCTGCAG G-3′,5′-CCACAG C-3′ and so on. The branch point is an “A ” nucleotide located within the sequences homologous to 5′-TACT(A/T)A*(C/T)(-/C)-3′ (while the symbol “*” marks the branch site) (SEQ.ID.NO.3), including but not limited, 5′-TACTAAC-3′,5′-TACTTATC-3′ and so on. The poly-pyrimidine tract is a high T and/or C content oligonucleotide sequences homologous to 5′-(TY)m(C/-)(T)nC(C/-)-3′ or 5′-(TC)nNCTAG(G/-)-3′ (while Y is a C or T/U and the “−” means an empty site). The symbols of “m” and “n” indicates multiple repeats ≧1; most preferably, m=1˜3 and n=7˜12. For all the above splicing components, the deoxythymidine (T) nucleotide in a gene DNA template will be replaced by uridine (U) after RNA transcription.

To test the function of a spliced intron, various inserts were cloned into the SpRNAi through multiple restriction/cloning sites, preferably containing restriction sites for AatII, AccI, AflII/III, AgeI, ApaI/LI, AseI, Asp718I, BamHI, BbeI, BclI/II, BglII, BsmI, Bsp120I, BspHI/LU11I/120I, BsrI/BI/GI, BssHII/SI, BstBI/U1/XI, ClaI, Csp6I, DpnI, DraI/II, EagI, Ecl136II, EcoRI/RII/47III, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu10I, PstI, PvuI/II, RsaI, SacI/II, SalI, Sau3AI, SmaI, SnaBI, SphI, SspI, StuI, TaiI, TaqI, XbaI, XhoI and/or XmaI endonucleases. These intron inserts are DNA templates encoding aberrant RNAs selected from the group consisting of short-temporary RNA (stRNA), small-interfering RNA (siRNA), short-hairpin RNA (shRNA), microRNA (mRNA), double-stranded RNA (ds RNA), long deoxyribonucleotide-containing RNA (D-RNA) and potentially ribozyme RNA in either sense, antisense or both orientations. Based on current studies, the gene silencing effect of a hairpin-RNA-containing SpRNAi was stronger than that of sense- and antisense-RNA-containing SpRNAi, showing an average of >80% knockdown specificity to all targeted gene products. Such knockdown specificity is determined by the homologous or complementary region of an insert to the targeted gene transcript. For example, the tested hairpin-SpRNAi insert possessed about 40˜42% homology and another 40˜42% complementarity to the targeted gene domain, with-in-between of which an A/T-rich linker sequence filled in the rest 8˜10% space. To the less potent sense- and antisense-SpRNAi inserts, although the homology or complementarity can be increased up to 100%, an average of 40˜50% knockdown efficacy was detected in most of current transfection tests. Therefore, we can use the transfection of these different types of SpRNAi inserts and/or the combination thereof to manipulate specific gene expression levels of interest in cells.

Simultaneous Expression of rGFP and Silencing of eGFP by SpRNAi Transfection

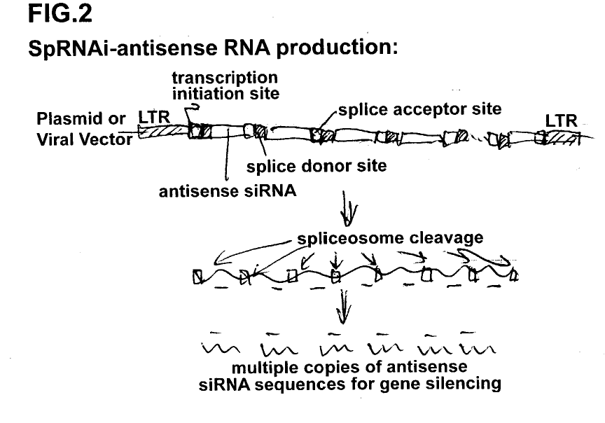

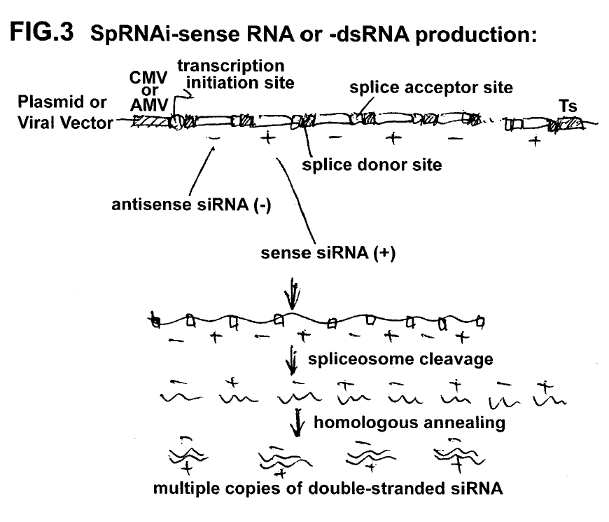

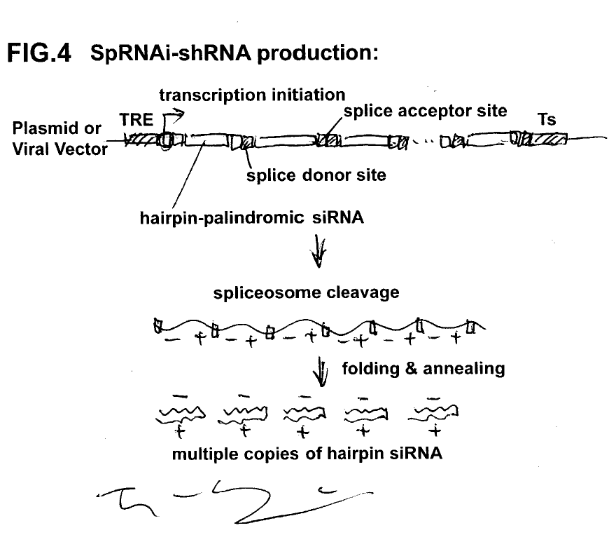

For the convenience of gene delivery and activation in tested cells, SpRNAi-inserted genes was preferably cloned into an expression-competent vector, selected from the group consisting of plasmid, cosmid, phagmid, yeast artificial chromosome, viral vectors and so on. As shown in FIGS. 1 and 2-4, the vectors contain at least one viral or type-II RNA polymerase (Pol-II) promoter or both for expressing of the SpRNAi-gene in eukaryotic cells, a Kozak consensus translation initiation site to increase translation efficiency in eukaryotic cells, SV40 polyadenylation signals downstream of the SpRNAi-gene for processing of the 3′-end gene transcript, a pUC origin of replication for propagation in prokaryotic cells, at least two multiple restriction/cloning sites for cloning of the SpRNAi-gene, an optional SV40 origin for replication in mammalian cells expressing the SV40 T antigen and an optional SV40 early promoter for expressing antibiotic resistance gene in replication-competent prokaryotic cells. The expression of antibiotic resistance genes is used to serve as a selective marker for searching of successfully transfected or infected clones, possessing resistance to the antibiotics selected from the group consisted of penicillin G, ampcillin, neomycin, paromycin, kanamycin, streptomycin, erythromycin, spectromycin, phophomycin, tetracycline, rifapicin, amphotericin B, gentamicin, chloramphenicol, cephalothin, tylosin and the combination thereof. The vector will be therefore stable enough to be introduced into a cell(s), tissue or animal body by a high efficient gene delivery method selected from the group consisting of liposomal transfection, chemical transfection, chemical transformation, electroporation, infection, micro-injection and gene-gun penetration.

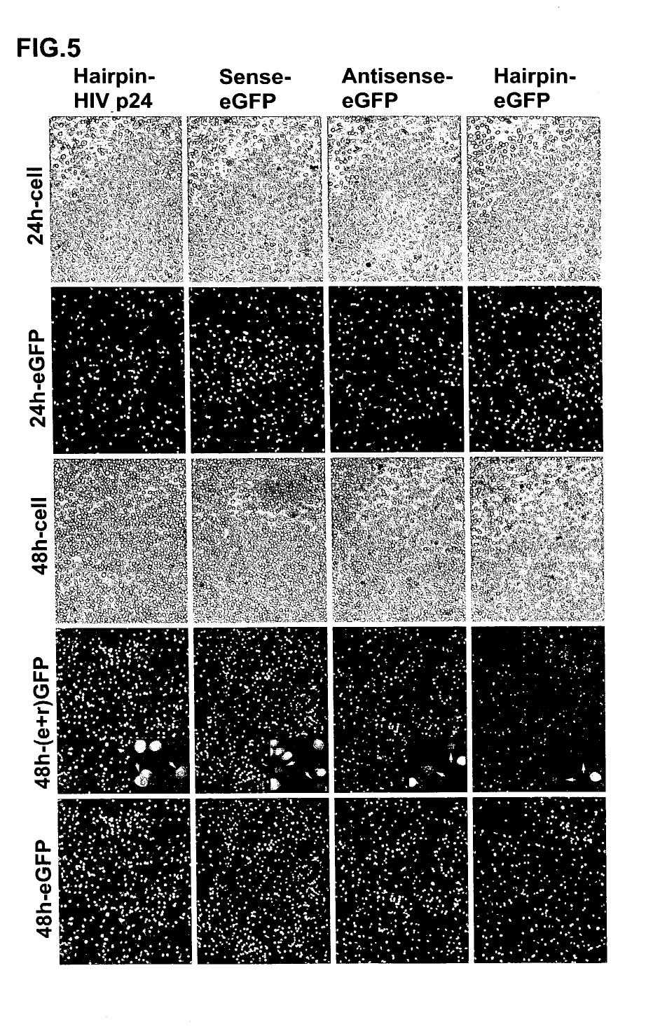

As shown in FIG. 5, the transfection of the pre-designed plasmids made from Examples 2 and 3 containing various SpRNAi-rGFP recombinant genes against the expression of a commercially available Aequorea Victoria green fluorescent protein (eGFP) was found to be successful in both expression of rGFP (red) and silencing eGFP (green). The use of eGFP-positive rat neuronal stem cell clones provided an excellent visual aid to measure the silencing effects of various SpRNAi inserts. Rat neuronal stem cell clones AP31 and PZ5a were primary cultured and maintained as described in Example 1. Observing from the cell culture after 24-h transfection, almost the same amount of total cell number and eGFP-positive cell population were well seeded and very limited apoptotic or differentiated cells occurred. Silencing of eGFP emission was detected at the 518-nm wavelength 36˜48 hours after transfection, indicating a potential onset timing required for the release of small interfering inserts from SpRNAi-rGFP gene transcripts by spliceosome machinery. Since all successfully transfected cells displayed red fluorescent emission at about 570-nm wavelength, we were able to trace the gene silencing effect by measuring relative light intensity of the green fluorescent emission in the red fluorescent cells, showing a knockdown potency of hairpin-eGFP>>sense-eGFP≈antisense-eGFP>>hairpin-HIV p24 (negative control) inserts.

Western Analysis of RNA Splicing/Processing-Directed eGFP Silencing Effects (n=3.

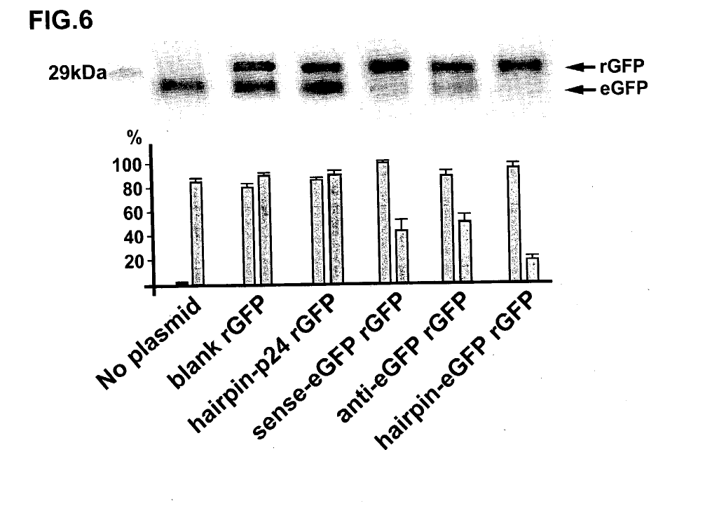

As shown in FIG. 6, quantitative knockdown levels of eGFP protein in the rat neuronal stem cell clones AP31 and PZ5a by various SpRNAi inserts were measured on a unreduced 6% SDS-polyacrylamide gel. For normalizing the loading amounts of transfected cellular proteins, rGFP protein levels (˜30 kDa; red bars) were adjusted to be comparatively equal, representing an average expression range from 82 to 100% intensity (Y axis). The eGFP levels (27 kDa; green bars) were found to be reduced by the transfection of SpRNAi-rGFP genes containing sense-eGFP (43.6%), antisense-eGFP (49.8%) and hairpin-eGFP (19.0%) inserts, but not that of intron-free rGFP gene (blank control) or SpRNAi-rGFP gene containing hairpin-HIV p24 insert (negative control). These findings confirm the above knockdown potency of hairpin-eGFP>>sense-eGFP antisense-eGFP>>hairpin-HIV p24 (negative control), and also demonstrate that only a gene insert which is either homologous or complementary (or both partially) to the targeted gene can elicit this gene-specific gene silencing effects.

Western Analysis of RNA Splicing/Processing-Directed Integrin β1 Silencing Effects in Human Prostatic Cancer LNCaP Cells (n=3).

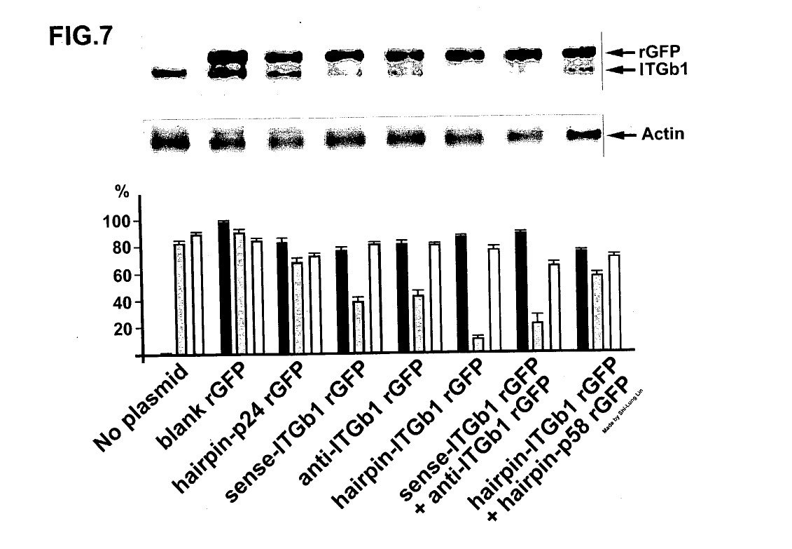

As shown in FIG. 7, a similar splicing/processing-directed gene silencing phenomenon was seen in human cancerous LNCaP cells. Quantitative knockdown levels of integrin β1 (ITGb1) protein by various SpRNAi inserts were measured on a reduced 8% SDS-polyacrylamide gel. The relative amounts of rGFP (black bars), ITGb1 (gray bars) and actin (white bars) were shown by a percentage scale (Y axis). The ITGb1 levels (29 kDa) were significantly reduced by the transfection of SpRNAi-rGFP genes containing sense-ITGb1 (37.3%), antisense-ITGb1 (48.1%) and hairpin-ITGb1 (13.5%) inserts, but not that of intron-free rGFP gene (blank control) or SpRNAi-rGFP gene containing hairpin-HIV p24 insert (negative control). Co-transfection of SpRNAi-rGFP genes containing sense- and antisense-ITGb1 inserts elicited a significant gene silencing effect (22.5%) in company with 10˜15% cell death, while that of SpRNAi-rGFP genes containing hairpin-ITGb1 and hairpin-p58/HHR23 inserts partially blocked the splicing-directed gene silencing effect to achieve an average 57.8% expression level. These findings indicate that the SpRNAi-induced gene silencing effects may work on a wide range of genes and cell types of interest.

Potential Strategy for HIV Vaccination Using SpRNAi.

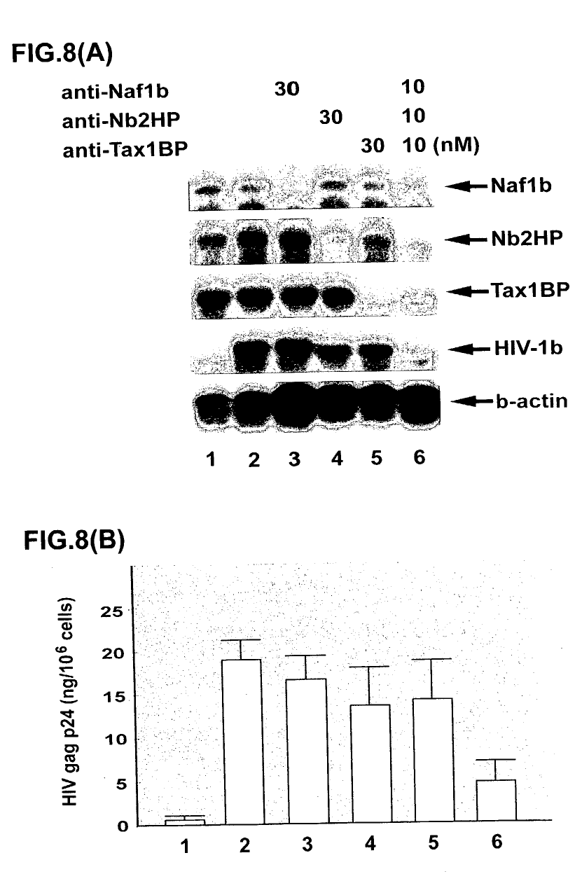

Northern analysis of SpRNAi silencing against HIV-promoted cellular genes is proven (FIG. 8). Feasibility of AIDS vaccination using SpRNAi products against cellular genes as anti-HIV drugs. FIG. 8A, Northern blot analysis of SpRNAi-induced gene silencing effects on Naf1β, Nb2HP and Tax1BP was shown to prevent HIV-1 type B infection. The tested gene targets were selected through RNA-PCR microarray analysis of differential expression genes from the acute (one˜two week) and chronic (about two year) infected patients' primary T cells with or without 25 nM anti-HIV D-RNAi treatment (Lin et.al. (2001) supra). The SpRNAi product concentrations of all treatments were normalized to 30 nM in total. FIG. 8B displays the bar chart of HIV-gag p24 ELISA results (white) in correlation to the treatment results of FIG. 8A.

In view of CD4 function in IL-2 stimulation and T-cell growth and activation, CD4 may not be an ideal target for HIV prevention. However, the search for mV-dependent cellular genes in vivo was hindered by the fact that infectivity of viruses and infection rate among different patients are usually different leading to inconsistent results. Short-term ex-vivo culture conditions can normalize infectivity and infection rate of HIV transmission in a more uniform CD4⁺ T cell population. Microarray analysis based on such ex vivo conditions would be reliable for critical biomedical and genetic research of HIV-1 infection. Our studies of microarray-identified differential gene profiles between HIV⁻ and HIV⁺ T cells in the acute and chronic infection phases has provided many potential anti-HIV cellular gene targets for AIDS therapy and prevention. To functionally evaluate the usefulness of targeting cellular genes for HIV vaccination, three highly differentially expressed genes, Naf1β, Nb2 homologous protein to Wnt-6 (Nb2HP) and Tax1 binding protein (Tax1BP), has been tested to inhibit HIV-1 infectivity. Because each of them contributes only parts of AIDS complications, knockdown of single target gene failed to suppress HIV-1 infection, while combination of all three SpRNAi probes at the same total concentration showed a significant 80±10% reduction of HIV-1b infection (FIG. 8A, n=3, p<0.01). The relative ELISA results of HIV gag-p24 protein (FIG. 8B) also correlated with the Northern blot data, showing an average of 77±5% reduction of gag-p24 expression. These findings indicate the feasibility of a novel strategy for retroviral vaccination using PTGS mechanisms against cellular target genes.

Marked Differences Between Traditional RNAi and Current SpRNAi

We found two major phenomenal differences between PTGS/RNAi and SpRNAi mechanisms. First, the onset of SpRNAi effects takes a period of time more than 36-48 hours, which is longer than the timing needed for the onset of PTGS/RNAi (12-24 hours). Second, although the function of PTGS/RNAi-associated Dicer enzymes is unclear to the SpRNAi-directed gene silencing mechanism, several repair complementing antigens have been found to be involved instead. Homologous recombination machinery involving nucleotide excision repair-related gene p58/HHR23 were identified to play a potential role of Dicer in SpRNAi induction. The p58/HHR23 species that codes for XP-C repair-complementing proteins is a human homologue of yeast RAD23 derivatives sharing an ubiquitin-like N-terminus. Based on its molecular similarity shared with RNA repairing-directed transcription regulation, the repair-complementing machinery may be able to reveal a novel mechanism of posttranscriptional gene silencing in addition to RNA interference.

A. Definitions

To facilitate understanding of the invention, a number of terms are defined below:

Nucleotide: a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. A nucleoside containing at least one phosphate group bonded to the 3′ or 5′ position of the pentose is a nucleotide.

Oligonucleotide: a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

Nucleic Acid: a polymer of nucleotides, either single or double stranded.

Nucleotide Analog: a purine or pyrimidine nucleotide that differs structurally from A, T, G, C, or U, but is sufficiently similar to substitute for the normal nucleotide in a nucleic acid molecule.

Gene: a nucleic acid whose nucleotide sequence codes for an RNA and/or a polypeptide (protein). A gene can be either RNA or DNA.

Base Pair (bp): a partnership of adenine (A) with thymine (T), or of cytosine (C) with guanine (G) in a double stranded DNA molecule. In RNA, uracil (U) is substituted for thymine. Generally the partnership is achieved through hydrogen bonding.

Intron: a part or parts of a gene sequence encoding non-protein reading frames.

Exon: a part or parts of a gene sequence encoding protein reading frames.

cDNA: a single stranded DNA that is homologous to an mRNA sequence and does not contain any intronic sequences.

Sense: a nucleic acid molecule in the same sequence order and composition as the homolog mRNA. The sense conformation is indicated with a “+”, “s” or “sense” symbol.

Antisense: a nucleic acid molecule complementary to the respective mRNA molecule. The antisense conformation is indicated as a “−” symbol or with a “a” or “antisense” in front of the DNA or RNA, e.g., “aDNA” or “aRNA”.

5′-end: a terminus lacking a nucleotide at the 5′ position of successive nucleotides in which the 5′-hydroxyl group of one nucleotide is joined to the 3′-hydroyl group of the next nucleotide by a phosphodiester linkage. Other groups, such as one or more phosphates, may be present on the terminus.

3′-end: a terminus lacking a nucleotide at the 3′ position of successive nucleotides in which the 5′-hydroxyl group of one nucleotide is joined to the 3′-hydroyl group of the next nucleotide by a phosphodiester linkage. Other groups, most often a hydroxyl group, may be present on the terminus.

Template: a nucleic acid molecule being copied by a nucleic acid polymerase. A template can be single-stranded, double-stranded or partially double-stranded, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

Nucleic Acid Template: a double-stranded DNA molecule, double stranded RNA molecule, hybrid molecules such as DNA-RNA or RNA-DNA hybrid, or single-stranded DNA or RNA molecule.

Conserved: a nucleotide sequence is conserved with respect to a preselected (reference) sequence if it non-randomly hybridizes to an exact complement of the preselected sequence.

Complementary or Complementarity or Complementation: used in reference to polynucleotides (i.e., a sequence of nucleotides) related by the base-pairing rules. For example, the sequence “A-G-T” is complementary to the sequence “T-C-A,” and also to “T-C-U.” Complementation can be between two DNA strands, a DNA and an RNA strand, or between two RNA strands. Complementarity may be “partial” or “complete” or “total”. Partial complementarity or complementation occurs when only some of the nucleic acid bases are matched according to the base pairing rules. Complete or total complementarity or complementation occurs when the bases are completely matched between the nucleic acid strands. The degree of complementarity between nucleic acid strands has significant effects on the efficiency and strength of hybridization between nucleic acid strands. This is of particular importance in amplification reactions, as well as in detection methods that depend on binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

Homologous or homology: refers to a polynucleotide sequence having similarities with a gene or mRNA sequence. A nucleic acid sequence may be partially or completely homologous to a particular gene or mRNA sequence, for example. Homology may also be expressed as a percentage determined by the number of similar nucleotides over the total number of nucleotides.

Complementary Bases: nucleotides that normally pair up when DNA or RNA adopts a double stranded configuration.

Complementary Nucleotide Sequence: a sequence of nucleotides in a single-stranded molecule of DNA or RNA that is sufficiently complementary to that on another single strand to specifically hybridize between the two strands with consequent hydrogen bonding.

Hybridize and Hybridization: the formation of complexes between nucleotide sequences which are sufficiently complementary to form complexes via complementary base pairing. Where a primer (or splice template) “hybridizes” with target (template), such complexes (or hybrids) are sufficiently stable to serve the priming function required by a DNA polymerase to initiate DNA synthesis. There is a specific, i.e. non-random, interaction between two complementary polynucleotide that can be competitively inhibited.

RNase H: an enzyme that degrades the RNA portion of an RNA/DNA duplex. RNase H may be an endonuclease or an exonuclease. Most reverse transcriptase enzymes normally contain an RNase H activity. However, other sources of RNase H are available, without an associated polymerase activity. The degradation may result in separation of the RNA from a RNA/DNA complex. Alternatively, the RNase H may simply cut the RNA at various locations such that pieces of the RNA melt off or are susceptible to enzymes that unwind portions of the RNA.

Vector: a recombinant nucleic acid molecule such as recombinant DNA (rDNA) capable of movement and residence in different genetic environments. Generally, another nucleic acid is operatively linked therein. The vector can be capable of autonomous replication in a cell in which case the vector and the attached segment is replicated. One type of preferred vector is an episome, i.e., a nucleic acid molecule capable of extrachromosomal replication. Preferred vectors are those capable of autonomous replication and/or expression of nucleic acids to which they are linked. Vectors capable of directing the expression of genes encoding for one or more polypeptides are referred to herein as “expression vectors”. Particularly important vectors allow cloning of cDNA from mRNAs produced using a reverse transcriptase.

Cistron: a sequence of nucleotides in a DNA molecule coding for an amino acid residue sequence and including upstream and downstream DNA expression control elements.

Promoter: a nucleic acid to which a polymerase molecule recognizes, perhaps binds to, and initiates synthesis. For the purposes of the instant invention, a promoter can be a known polymerase binding site, an enhancer and the like, any sequence that can initiate synthesis by a desired polymerase.

Antibody: a peptide or protein molecule having a preselected conserved domain structure coding for a receptor capable of binding a preselected ligand.

B. Compositions

A recombinant nucleic acid composition for inducing of RNA splicing/processing-associated gene silencing comprises:

a) At least an intron, wherein said intron is flanked with a plurality of exons and can be cleaved out of the exons by cellular RNA splicing and/or processing machinery; and

b) A plurality of exons, wherein said exons can be linked to form a gene possessing desired function.

The above recombinant nucleic acid composition, further comprises:

a) At least a multiple restriction/cloning site, wherein said multiple restriction/cloning site is used for ligation with an expression-competent vector for expressing of the RNA transcript of said recombinant nucleic acid composition; and

b) A plurality of transcription and/or translation termination sites, wherein said transcription and/or translation termination sites are used for produce the correct RNA transcript sizes of said recombinant nucleic acid composition.

The intron of the above recombinant nucleic acid composition, further comprises:

a) A gene-specific homologous insert;

b) A splicing donor site;

c) A splicing acceptor site;

d) A branch point domain for splicing recognition;

e) At least a poly-pyrimidine tract for spliceosome interaction; and

f) A plurality of linkers for connection of the above major components.

Based on prior studies, the splicing donor site is an oligonucleotide sequence either containing or homologous to the (5′-exon-AG)-(splicing point)-GTA(A/-)GAG(G/T)-3′ sequences (SEQ.ID.NO.1), including but not limited, 5′-AG GTAAGAGGAT-3′,5′-AG GTAAGAGT-3′,5′-AG GTAGAGT-3′,5′-AG GTAAGT-3′ and so on. The splicing acceptor site is an oligonucleotide sequence either containing or homologous to 5′-G(W/-)(T/G)(C/G)C(T/C)(G/A)CAG-(splicing point)-(G/C-3′-exon) sequences (while W is a pyrimidine A, T or U) (SEQ.ID.NO.2), including but not limited, 5′-GATATCCTGCAG G-3′,5′-GGCTGCAG G-3′,5′-CCACAG C-3′ and so on. The branch point is an “A ”nucleotide located within the sequences homologous to 5′-TACT(A/T)A*(C/T)(-/C)-3′ (while the symbol “*” marks the branch site) (SEQ.ID.NO.3), including but not limited, 5′-TACTAAC-3′,5′-TACTTATC-3′ and so on. The poly-pyrimidine tract is a high T or C content oligonucleotide sequences homologous to 5′-(TY)m(C/-)(T)nC(C/-)-3′ or 5′-(TC)n NCTAG(G/-)-3′ (while Y is a C or T/U and the “−” means an empty site). The symbols of “m” and “n” indicates multiple repeats ≧1; most preferably, m=1˜3 and n=7˜12. For all the above splicing components, the deoxythymidine (T) nucleotide in the intron of said recombinant nucleic acid composition is replaced by uridine (U) after RNA transcription.

C. Methods

A method for inducing of RNA splicing/processing-associated gene silencing effects comprises:

a) Constructing a recombinant nucleic acid composition containing at least an intron flanked with a plurality of exons, wherein said intron can be cleaved out of the exons by RNA splicing and/or processing for gene silencing and said exons can be linked together to form a gene with desired function;

b) Cloning said recombinant nucleic acid composition into an expression-competent vector;

c) Introducing said vector into a cell or in vivo;

d) Generating RNA transcript of said recombinant nucleic acid composition; and

e) Releasing the metabolic products of said intron by RNA splicing/processing mechanisms, so as to provide gene silencing effects against the genes containing sequences homologous to said intron.

Alternatively, a method for inducing of posttranscriptional gene silencing effects comprises:

a) Constructing a recombinant gene containing a functional RNA polymerase promoter and at least an intron flanked with a plurality of exons, wherein said intron can be cleaved out of the exons by RNA splicing and/or processing for gene silencing and said exons can be linked together to form a gene with desired function;

b) Introducing said recombinant gene into a cell cells, tissue or in vivo;

c) Generating RNA transcript of said recombinant gene; and

d) Releasing the metabolic products of said intron by RNA splicing/processing mechanisms, so as to provide gene silencing effects against the genes containing sequences homologous to said intron.

### EXAMPLES

The following examples serve to illustrate certain preferred embodiments and aspects of the present invention and are not to be construed as limiting the scope thereof.

In the experimental disclosure which follows, the following abbreviations apply: M (molar); mM (millimolar); μm (micromolar); mol (moles); pmol (picomolar); gm (grams); mg (milligrams); L (liters); ml (milliliters); μl (microliters); ° C. (degrees Centigrade); cDNA (copy or complementary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); NaCl (sodium chloride); HEPES (N-2-hydroxyethylpiperazine-N-2-ethanesulfonic acid); HBS (HEPES buffered saline); SDS (sodium dodecylsulfate); Tris-HCl (tris-hydroxymethylaminomethane-hydrochloride); and ATCC (American Type Culture Collection, Rockville, Md.).

### Example 1

### Cell Culture and Treatments

Rat neuronal stem cell clones AP31 and PZ5a were primary cultured and maintained as described by Palmer et.al., (J. Neuroscience, 1999). The cells were grown on polyornathine/laminin-coated dishes in DMEM/F-12 (1:1; high glucose) medium containing 1 mM L-glutamine supplemented with 1×N2 supplements (Gibco/BRL, Gaithersburg, Md.) and 20 ng/ml FGF-2 (Invitrogen, Carlsbad, Calif.), without serum at 37° C. under 5% CO₂. For long-term primary cultures, 75% of the medium was replaced with new growth medium every 48 h. Cultures were passaged at ˜80% confluency by exposing cells to trypsin-EDTA solution (Irvine Scientific) for 1 min and rinsing once with DMEM/F-12. Detached cells were replated at 1:10 dilution in fresh growth medium supplemented with 30% (v/v) conditioned medium which had exposed to cells for 24 h before passaging. Human prostatic cancer LNCaP cells were obtained from the American Type Culture Collection (ATCC, Rockville, Md.) and grown in RPMI 1640 medium supplemented with 10% fetal bovine serum with 100 μg/ml gentamycin at 37° C. under 10% CO₂. The LNCaP culture was passaged at ˜80% confluency by exposing cells to trypsin-EDTA solution for 1 min and rinsing once with RPMI, and detached cells were replated at 1:10 dilution in fresh growth medium. After a 48-hour incubation period, RNA from tested cells was isolated by RNeasy spin columns (Qiagen, Valencia, Calif.), fractionated on a 1% formaldehyde-agarose gel, and transferred onto nylon membranes. The genomic DNA was also isolated by apoptotic DNA ladder kit (Roche Biochemicals, Indianapolis, Ind.) and assessed by 2% agarose gel electrophoresis, while cell growth and morphology were examined by microscopy and cell counting.

### Example 2

### SpRNAi-Containing Gene Construction

Synthetic nucleic acid sequences used for generation of three different SpRNAi introns containing either sense, antisense or hairpin eGFP insert were listed as followings: N1-sense, 5′-pGTAAGAGGAT CCGATCGCAG GAGCGCACCA TCTTCTTCAA GA-3′ (SEQ.ID.NO.4); N1-antisense, 5′-pCGCGTCTTGA AGAAGATGGT GCGCTCCTGC GATCGGATCC TCTTAC-3′ (SEQ.ID.NO.5); N2-sense, 5′-pGTAAGAGGAT CCGATCGCTT GAAGAAGATG GTGCGCTCCT GA-3′ (SEQ.ID.NO.6); N2-antisense, 5′-pCGCGTCAGGA GCGCACCATC TTCTTCAAGC GATCGGATCC TCTTAC-3′ (SEQ.ID.NO.7); N3-sense, 5′-pGTAAGAGGAT CCGATCGCAG GAGCGCACCA TCTTCTTCAA GTTAACTTGA AGAAGATGGT GCGCTCCTGA-3′ (SEQ.ID.NO.8); N3-antisense, 5′-pCGCGTCAGGA GCGCACCATC TTCTTCAAGT TAACTTGAAG AAGATGGTGC GCTCCTGCGA TCGGATCCTC TTAC-3′ (SEQ.ID.NO.9); N4-sense, 5′-pCGCGTTACTA ACTGGTACCT CTTCTTTTT TTTTTGATAT CCTGCAG-3′ (SEQ.ID.NO.10); N4-antisense, 5′-pGTCCTGCAGG ATATCAAAAA AAAAAGAAGA GGTACCAGTT AGTAA-3′ (SEQ.ID.NO.11). Additionally, two exon fragments were generated by DraII restriction enzyme cleavage of red fluorescent rGFP gene (SEQ.ID.NO.12) at its 208th nucleotide (nt) site and the 5′ fragment was further blunt-ended by T4 DNA polymerase. The rGFP referred to a new red-fluorescin chromoprotein generated by insertion of an additional aspartate at the 69th amino acid (aa) of HcRed1 chromoproteins from Heteractis crispa., developing less aggregate and almost twice intense far-red fluorescent emission at the ˜570-nm wavelength. This mutated rGFP gene sequence was cloned into pHcRed1-N1/1 plasmid vector (BD Biosciences) and propagated with E. coli DH5α LB-culture containing 50 μg/ml kanamycin (Sigma). We cleaved the pHcRed1-N1/1 plasmid with XhoI and XbaI restriction enzymes and purified a 769-bp rGFP fragment and a 3,934-bp empty plasmid separately from 2% agarose gel electrophoresis.

Hybridization of N1-sense to N1-antisense, N2-sense to N2-antisense, N3-sense to N3-antisense and N4-sense to N4-antisense was separately performed by heating each complementary mixture of sense and antisense (1:1) sequences to 94° C. for 2 min and then 70° C. for 10 min in 1×PCR buffer (e.g. 50 mM Tris-HCl, pH 9.2 at 25° C., 16 mM (NH₄)₂SO₄, 1.75 mM MgCl₂). Continuously, sequential ligation of either N1, N2 or N3 hybrid to the N4 hybrid was performed by gradually cooling the mixture of N1-N4, N2-N4 or N3-N4 (1:1) hybrids respectively from 50° C. to 10° C. over a period of 1 h, and then T₄ ligase and relative buffer (Roche) were mixed with the mixture for 12 h at 12° C., so as to obtain introns for insertion into exons with proper ends. After the rGFP exon fragments were added into the reaction (1:1:1), T4 ligase and buffer were adjusted accordingly to reiterate ligation for another 12 h at 12° C. For cloning the right sized recombinant rGFP gene, 10 ng of the ligated nucleotide sequences were amplified by PCR with rGFP-specific primers 5′-dCTCGAGCATG GTGAGCGGCC TGCTGAA-3′ (SEQ.ID.NO.13) and 5′-dTCTAGAAGTT GGCCTTCTCG GGCAGGT-3′ (SEQ.ID.NO.14) at 94° C., 1 min, 52°, 1 min and then 68° C., 2 min for 30 cycles. The resulting PCR products were fractionated on a 2% agarose gel, and a ˜900-bp nucleotide sequences was extracted and purified by gel extraction kit (Qiagen). The composition of this ˜900 bp SpRNAi-eGFP-containing rGFP gene was further confirmed by sequencing.

Because the recombinant gene possessed an XhoI and an XbaI restriction site at its 5′- and 3′-end respectively, it can be easily cloned into a vector with relatively complementary ends to the XhoI and XbaI cloning sites. The vector was an expressing-capable organism or suborganism selected from the group consisted of plasmid, cosmid, phagmid, yeast artificial chromosome and viral vectors. Moreover, since the insert within the intron was flanked with a PvuI and an MluI restriction site at its 5′- and 3′-end respectively, we can remove and replace the insert with another different insert sequence possessing relatively complementary ends to the PvuI and MluI cloning sites. The insert sequence was homologous or complementary to a gene fragment selected from the group consisted of fluorescent protein genes, luciferase genes, lac-Z genes, plant genes, viral genomes, bacterial genes, animal genes and human oncogenes. The homology and/or complementarity rate is ranged from about 30˜100%, more preferably 35˜49% for a hairpin-shRNA insert and 90˜100% for both sense-stRNA and antisense-siRNA inserts.

### Example 3

### Vector Cloning of SpRNAi-Containing Genes

For cloning into plasmids, since the SpRNAi-recombinant rGFP gene possessed an XhoI and an XbaI restriction site at its 5′- and 3′-end, respectively, it can be easily cloned into a vector with relatively complementary ends to the XhoI and XbaI cloning sites. We mixed the SpRNAi-recombinant rGFP gene and the linearized 3,934-bp empty pHcRed1-N1/1 plasmid at 1:16 (w/w) ratio, cooled the mixture from 65° C. to 15° C. over a period of 50 min, and then added T₄ ligase and relative buffer accordingly into the mixture for ligation at 12° C. for 12 h. This formed a SpRNAi-recombinant rGFP-expressing plasmid vector which can be propagated in E. coli DH5α LB-culture containing 50 μg/ml kanamycin. A positive clone was confirmed by PCR with rGFP-specific primers SEQ.ID.NO.13 and SEQ.ID.NO.14 at 94° C., 1 min and then 68° C., 2 min for 30 cycles, and further sequencing. For cloning into viral vectors, the same ligation procedure was performed except using a XhoI/XbaI-linearized pLNCX2 retroviral vector (BD Biosciences) instead. Since the insert within the SpRNAi intron was flanked with a PvuI and a MluI restriction site at its 5′- and 3′-end respectively, we removed and replaced the eGFP insert with various integrin β1-specific insert sequences possessing relatively complementary ends to the PvuI and MluI cloning sites.

Synthetic nucleic acid sequences used for generation of various SpRNAi introns containing either sense, antisense or hairpin integrin β1 insert were listed as followings: P1-sense, 5′-pCGCAAGCAGG GCCAAATTGT GGGTA-3′ (SEQ.ID.NO.15); P1-antisense, 5′-pTAGCACCCAC AATTTGGCCC TGCTTGTGCG C-3′ (SEQ.ID.NO. 16); P2-sense, 5′-pCGACCCACAA TTTGGCCCTG CTTGA-3′ (SEQ.ID.NO.17); P2-antisense, 5′-pTAGCCAAGCA GGGCCAAATT GTGGGTTGCG C-3′ (SEQ.ID.NO.18); P3-sense, 5′-pCGCAAGCAGG GCCAAATTGT GGGTTTAAAC CCACAATTTG GCCCTGCTTG A-3′ (SEQ.ID.NO. 19); P3-antisense, 5′-pTAGCACCCAC AATTTGGCCC TGCTTGAATT CAAGCAGGGC CAAATTGTGG GTTGCGC (SEQ.ID.NO.20). These inserts were designed using Gene Runner software v3.0 (Hastings, Calif.) and formed by hybridization of P1-sense to P1-antisense, P2-sense to P2-antisense and P3-sense to P3-antisense, targeting the 244˜265th-nt sequence of integrin β1 gene (NM 002211.2). The SpRNAi-containing rGFP-expressing retroviral vector can be propagated in E. coli DH5α: LB-culture containing 100 μg/ml ampcillin (Sigma). We also used a packaging cell line PT67 (BD Biosciences) for producing infectious, replication-incompetent virus. The transfected PT67 cells were grown in DMEM medium supplemented with 10% fetal bovine serum with 4 mM L-glutamine, 1 mM sodium pyruvate, 100 μg/ml streptomycin sulfate and 50 μg/ml neomycin (Sigma) at 37° C. under 5% CO₂. The titer of virus produced by PT67 cells was determined to be at least 10⁶ cfu/ml before transfection.

### Example 4

### Low Stringent Northern Blot Analysis

RNA (20 μg total RNA or 2 μg poly[A⁺] RNA) was fractionated on 1% formaldehyde-agarose gels and transferred onto nylon membranes (Schleicher&Schuell, Keene, N.H.). A synthetic 75-bp probe (5′-dCCTGGCCCCC TGCTGCGAGT ACGGCAGCAG GACGTAAGAG GATCCGATCG CAGGAGCGCA CCATCTTCTT CAAGT-3′ (SEQ.ID.NO.21)) targeting the junction region between rGFP and hairpin eGFP-insert was labeled with the Prime-It II kit (Stratagene, La Jolla, Calif.) by random primer extension in the presence of [³²P]-dATP (>3000 Ci/mM, Amersham International, Arlington Heights, Ill.), and purified with 30 bp-cutoff Micro Bio-Spin chromatography columns (Bio-Rad, Hercules, Calif.). Hybridization was carried out in the mixture of 50% freshly deionized formamide (pH 7.0), 5× Denhardt's solution, 0.5% SDS, 4×SSPE and 250 mg/mL denatured salmon sperm DNAs (18 h, 42° C.). Membranes were sequentially washed twice in 2×SSC, 0.1% SDS (15 min, 25° C.), and once in 0.2×SSC, 0.1% SDS (15 min, 25° C.) before autoradiography.

### Example 5

### Suppression of Specific Protein Expression Levels

For interference of eGFP expression, we transfected rat neuronal stem cells with SpRNAi-recombinant rGFP plasmids encoding either sense, antisense or hairpin eGFP insert, using Fugene reagent (Roche). Plasmids containing insert-free rGFP gene and SpRNAi-recombinant rGFP gene with an insert against HIV-gag p24 were used as negative control. Cell morphology and fluorescence imaging was photographed at 0-, 24- and 48-hour time points after transfection. At the 48-h incubation time point, the rGFP-positive cells were sorted by flow cytometry and collected for western blot analysis. For interference of integrin β1 expression, we transfected LNCaP cells with pLNCX2 retroviral vectors containing various SpRNAi-recombinant rGFP genes against the 244˜265th-nt domain of integrin β1 using the Fugene reagent. The transfection rate of pLNCX2 retroviral vector into LNCaP cells was tested to be about 20%, while the pLNCX2 virus was less infectious to LNCaP cells. The same analyses were performed as aforementioned.

### Example 6

### SDS-PAGE and Western Blot Analysis

For immunoblotting, cells were rinsed with ice cold PBS after growth medium was removed, and then treated with the CelLytic-M lysis/extraction reagent (Sigma Chemical, St. Louis, Mo.) supplemented with protease inhibitors, Leupeptin, TLCK, TAME and PMSF, following manufacture's recommendations. The cells were incubated at room temperature on a shaker for 15 min, scraped into microtubes, and centrifuged for 5 min at 12,000×g to pellet the cell debris. Protein-containing cell lysate were collected and stored at −70° C. until use. Protein determinations were prepared as described (Bradford, 1976), with SOFTmax software package on an E-max microplate reader (Molecular Devices, Sunnyvale, Calif.). Each 30 μg cell lysate was added into SDS-PAGE sample buffer either with (reduced) or without (unreduced) 50 mM DTT, and boiled for 3 min before loaded onto 8% polyacylamide gels, while the reference lane was loaded with 2˜3 μl molecular weight markers (BioRad). SDS-polyacrylamide gel electrophoresis was performed according to the standard protocols (Molecular Cloning, 3rd ED). Protein fractionations were electroblotted onto a nitrocellulose membrane, blocked with Odyssey blocking reagent (Li-Cor Biosciences, Lincoln, NB) for 1˜2 h at the room temperature. We assessed GFP expression using primary antibodies directed against eGFP (1:5,000; JL-8, BD Biosciences, Palo Alto, Calif.) or rGFP (1:10,000; BD Biosciences), overnight at 4° C. The blot was then rinsed 3 times with TBS-T and exposed to a secondary antibody, goat anti-mouse IgG conjugate with Alexa Fluor 680 reactive dye (1:2,000; Molecular Probes), for 1 h at the room temperature. After three more TBS-T rinses, scanning and image analysis were completed with Li-Cor Odyssey Infrared Imager and Odyssey Software v.10 (Li-Cor). For integrin β1 analysis, the same procedure was performed except using primary antibodies directed against integrin β1 (1:2,000; LM534, Chemicon, Temecula, Calif.).

### Example 7

### Combinational Therapy for HIV Eradication and Vaccination

The ex vivo transfection of a viral RNA-antisense DNA hybrid construct in conjunction with interleukin 2 adjuvant therapy has been found to silence average 99.8% human immunodeficiency virus-1 (HIV-1) subtype B gene activity through a novel posttranscriptional gene silencing mechanism, deoxyribonucleotidylated RNA interference (D-RNAi; Lin et.al. (2001) supra, which are herein incorporated as a reference). This combined therapy not only delivered a strong suppression effect to viral replication but also boosted the immunity and proliferation of non-infected CD4⁺ T lymphocytes. A normal T cell outgrowth effect was observed to achieve maximal 76.2% HIV-infected cell elimination after one-week therapy. RNA-directed endoribonuclease activity was mildly increased up to 6.7% by the transfection, while no interferon-induced cytotoxicity was detected. The cellular genes corresponding to combinational therapy have been further investigated by microarray analysis for AIDS prevention. Co-suppression of three microarray-identified target genes, Naf1β, Nb2 homologous protein to Wnt-6 and Tax1 binding protein was shown to prevent average 80.2% HIV-1b entry and infection in a primary CD4⁺ T cell model. These findings have lead to an immediate therapy in both acute and chronic HIV-1 infections and also a potential vaccination useful for AIDS elimination.

In order to test the effectiveness of D-RNAi to inactivate HIV-1 replication, a viral RNA (vRNA)-antisense DNA (aDNA) hybrid construct was designed to silence an early-stage gene locus containing gag/pol/pro viral genes and p24 HIV-1 gene marker. Expectedly, the anti-gag/pol/pro transfection will interfere the integration of viral provirus into host chromosome and also to prevent the activation of several viral genes, while the anti-p24 transfection will provide a visual indicator for observing viral activity on a ELISA assay. The results showed that such strategy was effective in knocking out exogenous viral gene expression ex vivo in a CD4⁺ T lymphocyte extract model. Peripheral blood mononuclear cells (PBMC) extracted from patients were purified by CD4⁺-affinity immunomagnetic beads and grown in RPMI 1640 medium with 200 U/ml IL-2 adjuvant treatment for more than two weeks. A vRNA-aDNA hybrid probe containing partial HIV genomic sequence from +2113 to +2453 bases was generated by a pre-designed SpRNAi-recombinant gene (as a control in previoue Examples) homologous to gag-p24 genes. After 96 h incubation, the expression activity of HIV-1 genome was measured by northern blotting and found to be almost completely shut down in the D-RNAi hybrid transfection sets.

As shown in Lin et.al (2001) Current Cancer Drug Targets 1: 241-247, the gene silencing effects of anti-HIV D-RNAi transfections in the acute phase AIDS patient T lymphocyte extracts were biostatistically significant (n=3, p<0.01). Pure HIV-1 provirus was shown as a viral genome sized about 9.7 kilo nucleotide bases on a formaldehyde-containing RNA electrophoresis gel. Samples of CD4⁺ Th lymphocyte RNA extracts from normal non-infected persons were used as negative control, while those from HIV-1 infected patients were used as positive control. No significant gene silencing effect was detected in all controls and transfections of other constructs, including vDNA-aRNA hybrid of HIV-1b, aDNA only and vRNA-aDNA against HIV-1 rather than HIV-1 region. In the acute phase (<2-week infection), the treatment of 5 nM D-RNAi transfections knocked out average 99.8% viral gene expression, whereas in the chronic phase (˜two-year infection), the same treatment knocked down only average 71.4% viral gene expression. Although higher RNase activities were found in chronic HIV-1⁺ T cells by microarray analysis, the transfection of higher concentrated D-RNAi more than 25 nM can overcome this drug resistance. Unlike dsRNA, the transfection of high concentrated vRNA-aDNA hybrids did not cause significant interferon-induced cytotoxic effects, because the house-keeping gene, β-actin, are expressed normally in all sets of cells. Because the Northern blot method is able to detect HIV-1 gene transcript at the nanogram level, the above strong viral gene silencing effect actually demonstrates a very promising pharmaceutical and therapeutical potential for the combinational treatments of D-RNAi and IL-2 as antiviral therapy and/or vaccination.

### Example 8

### In Vitro Deoxyribonucleotidylated RNA Probe Generation

The RNA-polymerase cycling reaction (RNA-PCR) procedure can be modified to synthesize mRNA-aDNA and/or mDNA-aRNA hybrids (Lin et.al. (1999) Nucleic Acids Res. 27, 4585-4589) from either SpRNAi-recombinant gene, expression-competent vector template or transcriptome source. As an example of using the SpRNAi-recombinant gene as a source, a SpRNAi-sense HIV recombinant gene containing homologues to HIV-1 genome from +2113 to +2453 bases was generated following a procedure similar to Example 2. The RNA products (10˜50 μg) of the SpRNAi-sense HIV recombinant gene were transcribed from about 10⁶ transfected cells, isolated by RNeasy columns (Qiagen) and then continuously hybrid to its pre-synthesized complementary DNA (cDNA) by heating and then cooling the mixture from 65° C. to 15° C. over a period of 50 min. Transfection was completed following the same procedure shown in Example 5.

### REFERENCES

The following references are hereby incorporated by reference as if fully set forth herein:

1. Elbashir et.al. (2001) Nature 411: 494-498.

2. Lin et.al. (2001) Biochem. Biophys. Res. Commun. 281: 639-644.

3. Grant S. (1999) “Dissecting the mechanisms of posttranscriptional gene silencing: divide and conquer”, Cell 96: 303-306.

4. Lin et.al (2001) Current Cancer Drug Targets 1: 241-247.

5. Stark et.al. (1998) Annu. Rev. Biochem. 67: 227-264.

6. Brantl S. (2002) Biochimica et Biophysica Acta 1575: 15-25.

7. Jen et.al. (2000) Stem Cells 18: 307-319.

8. Ying et.al. (1999) Biochem. Biophys. Res. Commun. 265: 669-673.

9. Tuschl et.al. (2002) Nat Biotechnol. 20: 446-448.

10. Miyagishi et.al. (2002) Nat Biotechnol 20: 497-500.

11. Lee et.al. (2002) Nat Biotechnol 20: 500-505.

12. Paul et.al. (2002) Nat Biotechnol 20: 505-508.

13. Geiduschek et.al. (2001) J. Mol Biol 310: 1-26.

14. Schramm et.al. (2002) Genes Dev 16: 2593-2620.

15. Clement et.al. (1999) RNA 5: 206-220.

16. Sittler et.al. (1987) J. Mol Biol 197: 737-741.

17. U.S. Pat. No. 4,289,850 to Robinson.

18. U.S. Pat. No. 6,159,714 to Lau.

It is understood that the examples and embodiments described herein are for illustrative purposes only and that various modifications or changes in light thereof will be suggested to persons skilled in the art, and are to be included within the spirit and purview of the invention as set forth in the appended claims. All publications and patents cited herein are incorporated herein by reference in their entirety for all purposes.

1. A method for inducing of RNA splicing/processing-associated gene silencing effects comprises the steps of:
(a) Constructing a recombinant nucleic acid composition containing at least an intron flanked with a plurality of exons, wherein said intron can be cleaved out of the exons by RNA splicing and/or processing for gene silencing and said exons can be linked together to form a gene with desired function;
(b) Cloning said recombinant nucleic acid composition into an expression-competent vector;
(c) Introducing said vector into a cell, cells, tissue or in vivo;
(d) Generating RNA transcript of said recombinant nucleic acid composition; and
(e) Releasing the metabolic products of said intron by RNA splicing/processing mechanisms, so as to provide gene silencing effects against the genes containing sequences homologous to said intron.

2. The method as defined in claim 1, further comprises the step of synthesizing the nucleic acid components of said intron or exon sequences, or both.

3. The method as defined in claim 1, further comprises the step of mixing a plurality of different kinds of said recombinant nucleic acid compositions between the step (a) and (b).

4. The method as defined in claim 1, further comprises the step of mixing a plurality of different kinds of said vectors between the step (b) and (c).

5. A method for inducing of posttranscriptional gene silencing effects comprises:
(a) Constructing a recombinant gene composition containing a functional RNA polymerase promoter and at least an intron flanked with a plurality of exons, wherein said intron can be cleaved out of the exons by RNA splicing and/or processing for gene silencing and said exons can be linked to form a gene with desired function;
(b) Introducing said recombinant gene composition into a cell or in vivo;
(c) Generating RNA transcript of said recombinant gene composition; and
(d) Releasing the metabolic products of said intron by RNA splicing/processing mechanisms, so as to provide gene silencing effects against the genes containing sequences homologous to said intron.

6. The method as defined in claim 5, further comprises the step of synthesizing the nucleic acid components of said intron or exon sequences, or both.

7. The method as defined in claim 5, further comprises the step of mixing a plurality of different kinds of said recombinant nucleic acid compositions between the step (a) and (b).

8. The method as defined in claims 1 and 5, wherein said intron is a nucleic acid sequence containing components selected from the group consisting of gene-homologous insert, branch point and poly-pyrimidine tract, and splicing donor and acceptor splicing sites.

9. The method as defined in claim 8, wherein said gene-homologous insert is a nucleic acid sequence containing components and/or analogs either homologous or complementary to at least a targeted gene selected from the group consisting of pathogenic nucleic acids, viral genes, mutated genes, oncogenes and many other types of functional as well as non-functional genes.

10. The method as defined in claim 8, wherein said gene-homologous insert is a nucleic acid template encoding aberrant RNAs selected from the group consisting of antisense RNA, short-temporary RNA (stRNA), small-interfering RNA (siRNA), short-hairpin RNA (shRNA), microRNA (mRNA), double-stranded RNA (dsRNA), long deoxyribonucleotide-containing RNA (D-RNA) and ribozyme RNA in either sense, antisense or both orientations.

11. The method as defined in claim 8, wherein said gene-homologous insert is a sense-oriented nucleic acid sequence containing about 40% to 100% homology to a targeted gene, most preferably containing about 90% to 100% homology to the targeted gene.

12. The method as defined in claim 8, wherein said gene-homologous insert is an antisense-oriented nucleic acid sequence containing about 40% to 100% homology to the complementary copy of a targeted gene, most preferably containing about 90% to 100% complementarity to the targeted gene.

13. The method as defined in claim 8, wherein said gene-homologous insert is a hairpin-like nucleic acid sequence containing about 35% to 65% homology and/or about 35% to 65% complementarity to a targeted gene, most preferably containing about 41 to 49% homology and about 41 to 49% complementarity to the targeted gene.

14. The method as defined in claim 8, wherein said gene-homologous insert is incorporated into said intron through at least a restriction/cloning site selected from the group consisting of AatII, AccI, AflII/III, AgeI, ApaI/LI, AseI, Asp718I, BamHI, BbeI, BclI/II, BglII, BsmI, Bsp120I, BspHI/LU11I/120I, BsrI/BI/GI, BssHII/SI, BstBI/U1/XI, ClaI, Csp6I, DpnI, DraI/II, EagI, Ecl136II, EcoRI/RII/47III, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu10I, PstI, PvuI/II, RsaI, SacI/II, SalI, Sau3AI, SmaI, SnaBI, SphI, SspI, StuI, TaiI, TaqI, XbaI, XhoI and/or XmaI cleavage domains.

15. The method as defined in claim 8, wherein said branch point is an adenosine (A) nucleotide located within a nucleic acid sequence containing or homologous to the 5′-TACTWAY-3′ sequences (SEQ.ID.NO.3).

16. The method as defined in claim 8, wherein said branch point is an adenosine (A) nucleotide located within a nucleic acid sequence containing at least an oligonucleotide selected from the group consisting of 5′-TACTAAC-3′ and 5′-TACTTATC-3′.

17. The method as defined in claim 8, wherein said poly-pyrimidine tract is a high T or C content oligonucleotide sequence containing or homologous to an oligonucleotide selected from the group consisting of 5′-(TY)m(C/-)(T)nC(C/-)-3′ and 5′-(TC)nNCTAG(G/-)-3′, while the symbols of “m” and “n” indicates multiple repeats ≧1; most preferably, the m number is equal to 1˜3 and the n number is equal to 7˜12.

18. The method as defined in claim 8, wherein said splicing donor site is a nucleic acid sequence either containing or homologous to the 5′-GTAAGAGK-3′ sequences (SEQ.ID.NO. 1).

19. The method as defined in claim 8, wherein said splicing donor site is a nucleic acid sequence containing or homologous to an oligonucleotide selected from the group consisting of 5′-AG GTAAGAGGAT-3′,5′-AG GTAAGAGT-3′,5′-AG GTAGAGT-3′ and 5′-AG GTAAGT-3′.

20. The method as defined in claim 8, wherein said splicing acceptor site is a nucleic acid sequence either containing or homologous to the GWKSCYRCAG sequences (SEQ.ID.NO.2).

21. The method as defined in claim 8, wherein said splicing acceptor site is a nucleic acid sequence containing or homologous to an oligonucleotide selected from the group consisting of 5′-GATATCCTGCAG G-3′,5′-GGCTGCAG G-3′ and 5′-CCACAG C-3′.

22. The method as defined in claim 1, wherein said vector is an expression-competent vector selected from the group consisting of plasmid, cosmid, phagmid, yeast artificial chromosome and viral vectors.

23. The method as defined in claim 1, wherein said vector contains at least a viral or type-II RNA polymerase (Pol-II) promoter or both, a Kozak consensus translation initiation site, polyadenylation signals and a plurality of restriction/cloning sites.

24. The method as defined in claim 23, wherein said restriction/cloning site is an oligonucleotide cleavage domain for at least an endonuclease selected from the group consisting of AatII, AccI, AflII/II, AgeI, ApaI/LI, AseI, Asp718I, BamHI, BbeI, BclI/II, BglII, BsmI, Bsp120I, BspHI/LU11I/120I, BsrI/BI/GI, BssHII/SI, BstBI/U1/XI, ClaI, Csp6I, DpnI, DraI/II, EagI, EagI, Ecl136II , EcoRI/RII/47II, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu10I, PstI, PvuI/II, RsaI, SacI/II, SalI, Sau3AI, SmaI, SnaBI, SphI, SspI, StuI, TaiI, TaqI, XbaI, XhoI and/or XmaI restriction enzymes.

25. The method as defined in claim 23, wherein said vector further contains a pUC origin of replication, a SV40 early promoter for expressing at least an antibiotic resistance gene in replication-competent prokaryotic cells and an optional SV40 origin for replication in mammalian cells.

26. The method as defined in claim 25, wherein said antibiotic resistance gene is selected from the group consisted of penicillin G, ampcillin, neomycin, paromycin, kanamycin, streptomycin, erythromycin, spectromycin, phophomycin, tetracycline, rifapicin, amphotericin B, gentamicin, chloramphenicol, cephalothin, tylosin and the combination thereof.

27. The method as defined in claims 1 and 5, wherein said recombinant composition is introduced into said cell or in vivo by a gene delivery method selected from the group consisting of liposomal transfection, chemical transfection, chemical transformation, homologous recombination, electroporation, infection, micro-injection and gene-gun penetration.

28. The method as defined in claims 1 and 5, wherein the RNA transcript of said recombinant composition is an ribonucleotide sequence selected from the group consisting of mRNA, hnRNA, rRNA, tRNA, viral RNA and their pre-RNA derivatives in either sense or antisense orientation.

29. The method as defined in claims 1 and 5, wherein the RNA transcript of said recombinant composition is generated by transcription machinery selected from the group consisting of type-II (Pol-II), type-I (Pol-I), type-III (Pol-III) and viral RNA polymerase transcription machinery.

30. The method as defined in claims 1 and 5, wherein said metabolic products of said intron is RNA selected from the group consisting of lariat-form RNA, antisense RNA, short-temporary RNA (stRNA), small-interfering RNA (siRNA), short-hairpin RNA (shRNA), microRNA (mRNA), aberrant RNA containing mis-matched conformation, double-stranded RNA (dsRNA), long deoxyribonucleotide-containing RNA (D-RNA) and ribozyme RNA in either sense, antisense or both orientations.

31. The method as defined in claims 1 and 5, wherein said metabolic products of said intron is released from said intron by a cleavage mechanism selected from the group consisting of RNA splicing, RNA processing and the combination thereof.

32. The method as defined in claims 1 and 5, wherein said gene silencing effect is caused by an intracellular mechanism selected from the group consisting of posttranscriptional gene silencing (PTGS), RNA interference (RNAi), ribozyme-associated RNA degradation, antisense- or mRNA-directed translation inhibition, gene replacement, RNA repairing and homologous complementing mechanisms.

33. The method as defined in claims 1 and 5, wherein the desired gene function of said exons is result from a genetic activity selected from the group consisting of normal gene expression, missing gene replacement, dominant-negative gene suppression, gene marker and targeting such as expression of fluorescent protein, luciferase, lac-Z and the derivatives as well as the combination thereof.

34. A medium containing said recombinant composition of claims 1 and/or 5 useful for disease prevention and treatment.

35. A recombinant nucleic acid composition for inducing of RNA splicing/processing-associated gene silencing comprises:
(a) At least an intron, wherein said intron is flanked with a plurality of exons and can be cleaved out of the exons by cellular RNA splicing and/or processing machinery; and
(b) A plurality of exons, wherein said exons can be linked to form a gene possessing desired function.

36. The composition as defined in claim 35, wherein said recombinant nucleic acid composition further comprises:
(a) At least a multiple restriction/cloning site, wherein said multiple restriction/cloning site is used for ligation with an expression-competent vector for expressing of the RNA transcript of said recombinant nucleic acid composition; and
(b) A plurality of transcription and/or translation termination sites, wherein said transcription and/or translation termination sites are used for produce the correct RNA transcript sizes of said recombinant nucleic acid composition.

37. The composition as defined in claim 35, wherein said intron of the recombinant nucleic acid composition comprises:
(a) An insert;
(b) A splicing donor site;
(c) A splicing acceptor site;
(d) A branch point domain for splicing recognition;
(e) At least a poly-pyrimidine tract for spliceosome interaction; and
(f) A plurality of nucleic acid linkers for connection of the above components.

38. An insert-containing intron composition, wherein the insert of said intron composition can be inserted into the intron area of a gene for producing of a desired RNA molecule through RNA splicing/processing mechanisms, comprises:
(a) An insert;
(b) A splicing donor site;
(c) A splicing acceptor site;
(d) A branch point domain for splicing recognition;
(e) At least a poly-pyrimidine tract for spliceosome interaction; and
(f) A plurality of nucleic acid linkers for connection of the above components.

39 The composition as defined in claims 37 and 38, wherein said insert is a nucleic acid sequence containing components and/or analogs either homologous or complementary to at least a targeted gene selected from the group consisting of pathogenic nucleic acids, viral genes, mutated genes, oncogenes and many other types of functional as well as non-functional genes.

40. The composition as defined in claims 37 and 38, wherein said insert is a nucleic acid template encoding aberrant RNAs selected from the group consisting of antisense RNA, short-temporary RNA (stRNA), small-interfering RNA (siRNA), short-hairpin RNA (shRNA), microRNA (mRNA), double-stranded RNA (dsRNA), long deoxyribonucleotide-containing RNA (D-RNA) and ribozyme RNA in either sense, antisense or both orientations.

41. The composition as defined in claims 37 and 38, wherein said insert is a sense-oriented nucleic acid sequence containing about 40% to 100% homology to a targeted gene, most preferably containing about 90% to 100% homology to the targeted gene.

42. The composition as defined in claims 37 and 38, wherein said insert is an antisense-oriented nucleic acid sequence containing about 40% to 100% homology to the complementary copy of a targeted gene, most preferably containing about 90% to 100% complementarity to the targeted gene.

43. The composition as defined in claims 37 and 38, wherein said insert is a hairpin-like nucleic acid sequence containing about 35% to 65% homology and/or about 35% to 65% complementarity to a targeted gene, most preferably containing about 41 to 49% homology and about 41 to 49% complementarity to the targeted gene.

44. The composition as defined in claims 37 and 38, wherein said insert is incorporated into said intron through at least a restriction/cloning site selected from the group consisting of AatII, AccI, AflII/III, AgeI, ApaI/LI, AseI, Asp718I, BamHI, BbeI, BclI/II, BglII, BsmI, Bsp120I, BspHI/LU11I/120I, BsrI/BI/GI, BssHII/SI, BstBI/U1/XI, ClaI, Csp6I, DpnI, DraI/II, EagI, Ecl136II, EcoRI/RII/47III, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu10I, PstI, PvuI/II, RsaI, SacI/II, SalI, Sau3AI, SmaI, SnaBI, SphI, SspI, StuI, TaiI, TaqI, XbaI, XhoI and/or XmaI cleavage domains.

45. The composition as defined in claims 37 and 38, wherein said branch point is an adenosine (A) nucleotide located within a nucleic acid sequence containing or homologous to the 5′-TACTWAY-3′ sequences (SEQ.ID.NO.3).

46. The composition as defined in claims 37 and 38, wherein said branch point is an adenosine (A) nucleotide located within a nucleic acid sequence containing at least an oligonucleotide selected from the group consisting of 5′-TACTAAC-3′ and 5′-TACTTATC-3′.

47. The composition as defined in claims 37 and 38, wherein said poly-pyrimidine tract is a high T or C content oligonucleotide sequence containing or homologous to an oligonucleotide selected from the group consisting of 5′-(TY)m(C/-)(T)nC(C/-)-3′ and 5′-(TC)nNCTAG(G/-)-3′, while the symbols of “m” and “n” indicates multiple repeats ≧1; most preferably, the m number is equal to 1˜3 and the n number is equal to 7˜12.

48. The composition as defined in claims 37 and 38, wherein said splicing donor site is a nucleic acid sequence either containing or homologous to the 5′-GTAAGAGK-3′ sequences (SEQ.ID.NO.1).

49. The composition as defined in claims 37 and 38, wherein said splicing donor site is a nucleic acid sequence containing or homologous to an oligonucleotide selected from the group consisting of 5′-AG GTAAGAGGAT-3′,5′-AG GTAAGAGT-3′, 5′-AG GTAGAGT-3′ and 5′-AG GTAAGT-3′.

50. The composition as defined in claims 37 and 38, wherein said splicing acceptor site is a nucleic acid sequence either containing or homologous to the GWKSCYRCAG sequences (SEQ.ID.NO.2).

51. The composition as defined in claims 37 and 38, wherein said splicing acceptor site is a nucleic acid sequence containing or homologous to an oligonucleotide selected from the group consisting of 5′-GATATCCTGCAG G-3′,5′-GGCTGCAG G-3′ and 5′-CCACAG C-3′.

52. The composition as defined in claim 35, wherein the desired gene function of said exons is result from a genetic activity selected from the group consisting of normal gene expression, missing gene replacement, dominant-negative gene suppression, gene marker and targeting such as expression of fluorescent protein, luciferase, lac-Z and the derivatives as well as the combination thereof.

53. A medium containing said composition of claims 35 and/or 38 useful for disease prevention and treatment.

54. A utilization of claims 8, 35 and 38, wherein said insert molecule is homologous to an RNA transcript or a part of the RNA transcript of a gene.

55. A utilization of claims 8, 35 and 38, wherein said insert molecule is in between about 17 to about 10,000 nucleotide bases, most preferably in between 19 to 2,000 bases.

56. A utilization of claims 8, 35 and 38, wherein said insert molecule is complementary to an RNA transcript or a part of the RNA transcript of a gene.

57. A utilization of claims 8, 35 and 38, wherein said insert molecule is sized between about 17 to about 10,000 nucleotide bases, most preferably in between 19 to 500 bases.
